(19) United States
(12) Patent Application Publication
(10) Pub. No.: US 2009/0027618 A1
(43) Pub. Date: Jan. 29, 2009

(54) Method and Arrangement for Automatic Detection and Interpretation of the Iris Structure for Determining Conditions of a Person

(76) Inventor: Petra Perner, Leipzig (DE)

Correspondence Address: GUDRUN E. HUCKETT DRAUDT, SCHUBERTSTR. 15A, WUPPERTAL, 42289, DE

(21) Appl. No.: 12/180,565

(22) Filed: Jul. 28, 2008

(30) Foreign Application Priority Data
Jul. 28, 2007 (DE) 10 2007 035 873.5

(51) Int. Cl. A61B 3/14; G06K 9/60

(52) U.S. Cl. 351/206; 351/246; 382/128

(57) ABSTRACT

For automatic detection and interpretation of the iris structure for determining a condition of a person, two-dimensional recording and digitization of an eyeball with iris is realized by a recording device connected to a data processing system; selection of the iris; generation of the topography of the iris by dividing the image of the iris into images of circular ring sections; determination of features for the circular ring sections and/or of objects in the images of the circular ring sections by combining image pixels of same color or intensity to individual objects by an object isolation method; feature determination of the objects determined by the object isolation method; comparison of the determined features of the objects with features saved as classification knowledge in the data processing system; and correlation of the determined objects to body regions in accordance with the topography of the iris are performed.

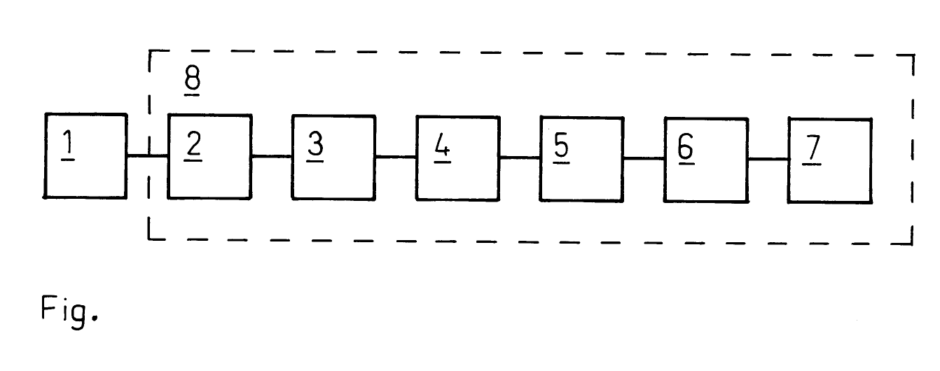

### BACKGROUND OF THE INVENTION

The invention relates to methods and arrangements for automatic detection and interpretation of the iris structure for determining conditions of a person.

The publication DE 20 2006 012 628 U1 (Device for detecting medical and/or biometric data of a living being) discloses a device for detecting, storing, comparing and evaluating medical features of a living being and/or biometric features that determine the individuality and thus the identity of a living being. This solution concerns recording, transmitting, and safe storing of such data. An evaluation of certain features of the images of the recordings is not provided.

The publication DE 10 2005 008 399 A1 (System of remote non-invasive diagnostics) concerns a system for the non-invasive remote diagnostics wherein by means of the optical state and a digital photo camera a transmission of the image of the iris is realized for determining the health condition of human beings by means of an automated method of iris diagnostics. The image of the recorded iris is transmitted to a computer for diagnostics. Diagnostics itself is realized manually by means of the represented image.

The present invention is based on the problem that the condition of a person is to be determined in a simple way by recognizing and interpreting the iris structure.

### SUMMARY OF THE INVENTION

This problem is solved for the method of the aforementioned kind by the following steps:

two-dimensional recording and digitalizing of an area of an eyeball together with the iris by means of a recording device connected to a data processing system,

selecting the iris,

generating the topography of the iris by dividing the image of the iris into images of circular ring sections,

determining features for the circular ring sections and/or objects in the images of the circular ring sections by combining image pixels of the same color or intensity to individual objects by means of an object isolation method,

determining features of the objects determined by the object isolation method,

comparing the determined features of the objects with features that are saved as classification knowledge in the data processing system, and

correlating the determined objects to body regions in accordance with the topography of the iris.

Moreover, the problem is solved for the arrangement of the aforementioned kind in that either a recording device that records two-dimensionally and digitalizes an area of the eyeball with the iris or a data memory with digitalized iris images is connected with at least one data processing system that receives the digitalized image in true colors or as a converted grayscale image. In the data processing system there are realized moreover

a device selecting the iris as a circular ring surface,

a device generating the topography of the iris by dividing the image of the iris into images of circular ring sections,

a device determining features for the circular ring sections and/or objects in the images of the circular ring sections by combining image pixels of same color or intensity to individual objects by means of an object isolation method,

a device determining features of the objects determined by the object isolation method,

a device comparing determined features of the objects with features that are saved as classification knowledge in the data processing system, and

a device assigning determined objects to body regions in accordance with the topography of the iris,

which devices are sequentially connected.

The iris is an aperture of the eye that is colored by pigments and is positioned at the transition from the cornea to the sclera. The iris regulates the quantity of light reaching the eye. The iris adjoins with the iris root the ciliary body and leaves open at its center an opening, the pupil. The iris is comprised of two layers. The front layer is the stroma that is differently designed in each human being. A pigment proportion in the stroma causes the iris to have a brown color, a low proportion causes it to be green to blue or grey. The rearward layer is the iris pigment layer. The pigment that is contained in the iris pigment layer provides filtering of scattered light.

Through the iris, by means of iridology, also referred to as iris analysis or iris diagnostics, the constitution, disposition regarding diseases, and the diseases themselves can be recognized. For this purpose, areas of the iris are topographically assigned to certain organs and body parts. By means of the structure of the iris and the assignment of the areas of the iris, the constitution, the disposition for diseases, and diseases themselves can be concluded.

The object of iridology or iris diagnostics is that by monitoring the condition and the change of the visible iris tissue information in regard to diseases or predisposition of the body can be derived. The basis for this is that, from the entire periphery with all organs, there are conducting paths leading to the iris. For this purpose, topographic charts of the iris are known from iridology and clinical testing of the iris signs. Advantageously, this is done automatically by means of the method and the arrangement after recording the iris.

Advantageously, this is furthermore achieved by means of a computer program with a program code either for performing the method for automatic detection and interpretation of the iris structure regarding a condition of a person when the program is running on a computer, and by a computer program product on a computer-readable memory either for performing the method for automatic detection and interpretation of the iris structure regarding the determination of a condition of a person when the program is running on a computer.

Advantageously, included are

a computer program with a program code for performing the method for automatic detection and interpretation of the iris structure for determining a condition of a person when the program is running on the computer, and

a computer program product on a computer-readable memory for performing the method for automatic detection and interpretation of the iris structure for determining a condition of person when the program is running on a computer. Advantageous embodiments of the invention are disclosed in the dependent claims.

In accordance with one embodiment of the invention, the iris as an iris collar in the form of a circular ring surface is selected and determined from the digitalized image by means of a circular mask for eliminating the pupil and a dot mask for the outer contour of the iris.

According to a further embodiment of the invention, the determined object in the image of the iris is represented on a screen connected to the data processing system and/or is stored, based on its position in the iris, in the memory of data processing system. In this way, a manual evaluation is simplified also.

According to a further embodiment of the invention, advantageously at least one of the features

object in the image of the circular ring segment is present or not,

number of objects,

surface area of the object,

average surface area of the object,

relative average surface area of the object,

average shape factor,

average contour length,

position of the object in the image of the circular ring section,

color or grayscale of the object, and

texture

is assigned to the determined object.

According to a further embodiment of the invention,

an unknown or uncertainly classified object is automatically recognized,

the object is represented on a data viewing device connected to the data processing system, and

this feature is correlated and assigned by means of a teaching unit automatically to the classification knowledge.

In accordance with a further embodiment, a teaching unit that assigns automatically an unknown or uncertainly classified object and a new feature determined therefrom to the classification knowledge is connected to the device comparing the features of the individual objects.

The teaching unit thus makes it possible that unknown features of the objects are acquired by evaluation through the user and/or operating personnel and automatically correlated and assigned to the saved and known features existing as classification knowledge in the data processing system.

### BRIEF DESCRIPTION OF THE DRAWING

An embodiment of the invention is illustrated in principle in the drawing and will be explained in the following with more detail.

The FIGURE shows an arrangement for automatic detection and interpretation of the iris structure for determining a condition of a person.

### DESCRIPTION OF PREFERRED EMBODIMENTS

In the following embodiment a method and an arrangement for automatic detection and interpretation of the iris structure for determining a condition of a person will be explained together in more detail.

The arrangement is comprised essentially either of a recording device 1 that records two-dimensionally and digitalizes an area of the eyeball with the iris or a data memory with iris recordings as well as at least one data processing system 8 that receives the digitalized image true to color or as a converted grayscale image. In the data processing system 8 there are realized

a device 2 selecting the iris as a circular ring surface,

a device 3 generating the topography of the iris by dividing the image of the iris into images of circular ring sections,

a device 4 determining features for the circular ring sections and/or objects in the images of the circular ring sections by combining image pixels of same color or intensity to individual objects by means of an object isolation method,

a device 5 determining features of the objects determined by the object isolation method,

a device 6 comparing the determined features of the objects with features that are saved as classification knowledge in the data processing system, and

a device 7 assigning determined objects to body regions in accordance with the topography of the iris.

The FIGURE shows an arrangement for automatic detection and interpretation of the iris structure for determining a condition of a person in a principal illustration.

By means of the recording device 1 a two-dimensional recording and digitalization of an area of an eyeball with the iris is performed. For this purpose, preferably a digital camera is provided wherein the image is advantageously taken by means of a microscope. The recording device 1 is connected to the data processing system 8.

Of course, it is also possible that a data memory with iris recordings can be connected to the data processing system 8. In this way, a local separation between recording device 1 and data processing system 8 is present.

In the data processing system 8 the devices 2 to 7 are sequentially coupled and connected.

In a device 2 that selects the iris as a circular ring surface, the iris is selected and determined as an iris collar in the form of a circular ring surface from the digitalized image by means of a circular mask for eliminating the pupil and a dot mask for the outer contour of the iris.

The topography of the iris is determined in the device 3 by dividing the image of the iris into images of circular ring segments. These circular ring segments are correlated to certain body parts.

Subsequently, a determination of the objects in the images of the circular ring sections is carried out. For this purpose, image points of same color or intensity are combined to individual objects and are determined by an object isolation method. This is done in the device 4 provided for this purpose.

Features of the objects determined by the object isolation method are determined in the device 5. In this connection, at least one of the features

object in the image of the circular ring segment is present or not,

number of objects,

surface area of the object,

average surface area of the object,

relative average surface area of the object,

average shape factor,

average contour length,

position of the object in the image of the circular ring section,

color or grayscale of the object, and

texture

is assigned to the determined object.

The determined features of the object are compared in the device 6 provided for this purpose with features saved in the data processing system 8 as classification knowledge

Subsequently, the determined objects are correlated by the device 7 to the body regions in accordance with the topography of the iris.

The determined object is represented in the image of the iris on a screen that is connected to the data processing system 8 and/or is saved, based on its position in the iris in the memory of the data processing system 8.

An unknown or uncertainly classified object is more over automatically detected, the object is represented by means of a data viewing device connected to the data processing system 8, and this feature is correlated and assigned by means of a teaching unit automatically to the classification knowledge.

For this purpose, a teaching unit that assigns automatically an unknown or uncertainly classified object and a new feature determined therefrom to the classification knowledge is connected to a device comparing the features of the individual objects.

The embodiment can moreover comprise

a computer program with a program code for performing the method for automatic detection and interpretation of the iris structure for determining a condition of a person when the program runs on a computer and

a computer program product on a computer-readable memory for performing the method for automatic detection and interpretation of the iris structure for determining a condition of a person when the program is running on a computer.

What is claimed is:

1-9. (canceled)

10. Method for automatic detection and interpretation of the iris structure for determining a condition of a person, the method comprising the steps of:
two-dimensionally recording and digitalizing as a digitalized image an area of an eyeball with the iris by a recording device connected to a data processing system;
selecting the iris as an iris image from said digitalized image of the eyeball;
determining a topography of the iris by dividing the iris image into images of circular ring sections;
determining in the images of said circular ring sections at least one individual object by combining image pixels of same color or intensity by an object isolation method;
determining features of said at least one individual object;
comparing said determined features of said at least one individual object with features saved in the data processing system as classification knowledge; and
correlating said at least one individual objects to body regions in accordance with said topography of the iris.

11. Method according to claim 10, wherein, in the step of selecting, said iris image is determined as an iris collar in the form of a circular ring surface by a circular mask that eliminates the pupil of the eyeball and a dot mask that determines the outer contour of the iris.

12. Method according to claim 10, wherein said at least one individual object is represented on a screen that is connected to the data processing system.

13. Method according to claim 10, wherein said at least one individual object is saved, based on its position in the iris, in the memory of the data processing system.

14. Method according to claim 10, wherein said at least one individual object is represented on a screen that is connected to the data processing system and is saved, based on its position in the iris, in the memory of the data processing system.

15. Method according to claim 10, wherein at least one feature selected from:
said at least one individual object is present or not,
number of said at least one individual object,
surface area of said at least one individual object,
average surface area of said at least one individual object,
relative average surface area of said at least one individual object,
average shape factor of said at least one individual object,
average contour length of said at least one individual object,
position of said at least one individual object in said images of said circular ring sections,
color or grayscale of said at least one individual object, and
texture of said at least one individual object
is assigned to said at least one individual object.

16. Method according to claim 10, further comprising the steps of automatically recognizing an unknown or uncertainly classified object, representing the unknown or uncertainly classified object on a data viewing device connected to the data processing system, and correlating and assigning unknown or uncertainly classified object by a teaching unit automatically to the classification knowledge.

17. Arrangement for automatic detection and interpretation of the iris structure for determining a condition of a person, the arrangement comprising:
a means that provides a digitalized image of an area of an eyeball with iris;
at least one data processing system connected to said device and receiving the digitalized image true to color or as a converted grayscale image;
wherein said at least one data processing system comprises:
a first device selecting from the digitalized image the iris as an iris image with a circular ring surface,
a second device generating a topography of the iris by dividing the iris image into images of circular ring sections,
a third device determining objects in the images of the circular ring sections by combining image pixels of same color or intensity to individual objects by an object isolation method,
a fourth device determining features of the individual objects,
a fifth device comparing the determined features of the individual objects with features saved as classification knowledge in the data processing system, and
a sixth device correlating the individual objects to body regions in accordance with the topography of the iris,
wherein said first, second, third, fourth, fifth, and sixth devices are sequentially coupled to one another.

18. Arrangement according to claim 17, wherein said means is a recording device recording two-dimensionally and digitalizing an area of the eyeball with the iris.

19. Arrangement according to claim 17, wherein said means is a data memory containing digitalized iris images.

20. Arrangement according to claim 17, further comprising a teaching unit that assigns automatically an unknown or uncertainly classified object and a new feature determined therefrom to the classification knowledge, wherein the teaching unit is connected to said fifth device.

21. Arrangement comprising at least one data processing system and a computer-readable memory coupled to the at least one data processing system, wherein the computer-readable memory contains program code that when executed implements a method for automatic detection and interpretation of the iris structure for determining a condition of a person, the method comprising the steps of:
two-dimensionally recording and digitalizing as a digitalized image an area of an eyeball with the iris by a recording device connected to a data processing system;
selecting the iris as an iris image from said digitalized image of the eyeball;
determining a topography of the iris by dividing the iris image into images of circular ring sections;
determining in the images of said circular ring sections at least one individual object by combining image pixels of same color or intensity by an object isolation method;
determining features of said at least one individual object;
comparing said determined features of said at least one individual object with features saved in the data processing system as classification knowledge; and
correlating said at least one individual objects to body regions in accordance with said topography of the iris.

22. Computer program product stored on a computer-readable memory for providing a program code for:
two-dimensionally recording and digitalizing as a digitalized image an area of an eyeball with the iris by a recording device connected to a data processing system;
selecting the iris as an iris image from said digitalized image of the eyeball;
determining a topography of the iris by dividing the iris image into images of circular ring sections;
determining in the images of said circular ring sections at least one individual object by combining image pixels of same color or intensity by an object isolation method;
determining features of said at least one individual object;
comparing said determined features of said at least one individual object with features saved in the data processing system as classification knowledge; and
correlating said at least one individual objects to body regions in accordance with said topography of the iris.
